(19) United States
(12) Patent Application Publication
(10) Pub. No.: US 2009/0204010 A1
(43) Pub. Date: Aug. 13, 2009

(54) Vein imaging apparatus

(75) Inventor: Makoto Mochizuki, Kawasaki (JP)

Correspondence Address: STAAS & HALSEY LLP, SUITE 700, 1201 NEW YORK AVENUE, N.W., WASHINGTON, DC, 20005, US

(73) Assignee: Fujitsu Limited, Kawasaki (JP)

(21) Appl. No.: 12/289,400

(22) Filed: Oct. 27, 2008

(30) Foreign Application Priority Data
Feb. 13, 2008 (JP) 2008-031204

(51) Int. Cl. A61B 5/00

(52) U.S. Cl. 600/476

(57) ABSTRACT

A vein imaging device that is capable of achieving space saving. The vein imaging device includes an imaging section and a guide member. The imaging section images a vein pattern of a palm. The guide member is erected on a periphery of an imaging surface of the imaging section. The guide member guides the position of the palm with respect to the imaging surface of the imaging section, by having a finger brought into contact therewith at a predetermined height position from the imaging surface of the imaging section.

### CROSS-REFERENCE TO RELATED APPLICATIONS

This application is based upon and claims the benefits of priority from the prior Japanese Patent Application No. 2008-031204, filed on Feb. 13, 2008, the entire contents of which are incorporated herein by reference.

### BACKGROUND OF THE INVENTION

1. Field of the Invention

This invention relates to a vein imaging device, and more particularly to a vein imaging device that images a vein pattern of a palm.

2. Description of the Related Art

With the recent development of a biometric authentication technology, it has been possible to identify individuals safely and accurately. Particularly, a method of authentication using veins of a palm or a finger is widely used from the viewpoint of its ease of use and high authentication ratio as a method of personal identification which ensures strong security, in a wide range from user authentication at ATMs (Automate Teller Machines) of financial institutions to login to personal computers.

Now, in the vein authentication, a pattern of veins, which is a body organ, is imaged using near infrared rays, and then the imaged vein pattern and a vein pattern registered in advance are compared with each other. Alternatively, the vein authentication is carried out by taking out and analyzing feature information from the imaged vein pattern, and performing a comparison using the feature information. This makes it necessary that a vein pattern or vein information provided which is registered, and a vein pattern or vein information imaged for personal identification accurately match each other. A major factor reducing the imaging accuracy of the vein pattern in the personal identification is a change in the position of a hand during imaging of the vein pattern. For example, if the hand is not positioned at a predetermined location suitable for imaging, or if the position of the hand is moved without being fixed, it can be difficult to accurately image the vein pattern.

To accurately image a vein pattern during personal identification, there has been proposed a method in which a vein pattern of the finger is imaged by fixing a finger as an object to be imaged to a predetermined location (see e.g. Japanese Laid-Open Patent Publication (Kokai) No. 2006-99493, and Japanese Laid-Open Patent Publication (Kokai) No. 2003-30632).

Further, when personal identification is performed by imaging a vein pattern of a palm and analyzing feature information thereon, it is necessary to dispose a whole hand as an object to be imaged, at a predetermined location, and then image the vein pattern.

FIGS. 12A and 12B are views of an example of a vein imaging device conventionally used for imaging a vein pattern of a palm. FIG. 12A is a left side view of the vein imaging device 50, while FIG. 12B is a top view of the vein imaging device 50. It should be noted that in FIG. 12A, a hand 54 is also illustrated which is placed on the vein imaging device 50 so as to image veins of the palm.

The vein imaging device 50 includes a support member 51, an imaging section 52, and a pair of guide members 53. The support member 51 supports the imaging section 52 and the guide members 53. The imaging section 52 is inserted into the support member 51. The imaging section 52 images a vein pattern of a palm of the hand 54. The pair of guide members 53 are erected with respect to an imaging surface of the imaging section 52. Each guide member 53 has a hand-holding portion 53a. The hand-holding portion 53a is provided for pacing the fingers and the wrist of the hand 54 thereon.

According to the vein imaging device 50 constructed as above, the hand 54 can be disposed at a predetermined location with respect to the imaging section 52, and hence it is possible to dispense with a function of increasing the angular field of view of a lens used in the imaging section 52, an autofocus function, and a function of correcting lens aberration. This makes it possible to realize an environment for personal identification based on a vein pattern at low costs.

In the vein imaging device 50, however, a region between the pair of hand-holding portion 53a is required to have an area covering a palm, so as to support the hand 54, so that an installation area of the vein imaging device 50 becomes dependent on the area of the region. This causes a problem that the vein imaging device 50 cannot be made compact in size even if the size of the imaging section 52 is reduced.

### SUMMARY OF THE INVENTION

The present invention has been made in view of these points, and an object thereof is to provide a vein imaging device which is space-saving.

To attain the above object, there is provided a vein imaging device that images a vein pattern of a palm. The vein imaging device is characterized in that it comprises an imaging section that images the vein pattern, and a guide member that is erected on a periphery of an imaging surface of the imaging section, and the guide member guides a position of the palm with respect to the imaging surface by having a finger brought into contact therewith at a predetermined height position from the imaging surface of the imaging section.

The above and other objects, features and advantages of the present invention will become apparent from the following description when taken in conjunction with the accompanying drawings which illustrate preferred embodiments of the present invention by way of example.

### BRIEF DESCRIPTION OF THE DRAWINGS

FIG. 1 is a perspective view of a vein imaging device according to a first embodiment of the present invention;

FIGS. 2A and 2B are views showing the construction of the vein imaging device according to the first embodiment, wherein FIG. 2A is a front view of the vein imaging device in use, and FIG. 2B is a cross-sectional view taken on line A-A of FIG. 2A;

FIG. 3 is a view showing a support member and a guide member of the vein imaging device according to the first embodiment, in a state in which they are separated from each other;

FIG. 4 is a schematic view showing an example of use of the vein imaging device according to the first embodiment;

FIG. 5 is a perspective view of a vein imaging device according to a second embodiment of the present invention;

FIGS. 6A and 6B are views showing the construction of the vein imaging device according to the second embodiment, wherein FIG. 6A is a left side view of the vein imaging device in use, and FIG. 6B is a top view of the same in storage;

FIG. 7 is a perspective view of a vein imaging device according to a third embodiment of the present invention;

FIGS. 8A and 8B are views showing the construction of the vein imaging device according to the third embodiment, wherein FIG. 8A is a right side view of the vein imaging device in use, and FIG. 8B is a top view of the same in storage;

FIG. 9 is a schematic view showing an example of use of the vein imaging device according to the third embodiment;

FIGS. 10A and 10B are views showing the construction of a vein imaging device according to a fourth embodiment, wherein FIG. 10A is a right side view of the vein imaging device in use, and FIG. 10B is a right side view of the same in storage;

FIG. 11 is a top view of the vein imaging device according to the fourth embodiment; and

FIGS. 12A and 12B are views of an example of a vein imaging device which is being used for imaging a vein pattern of a palm.

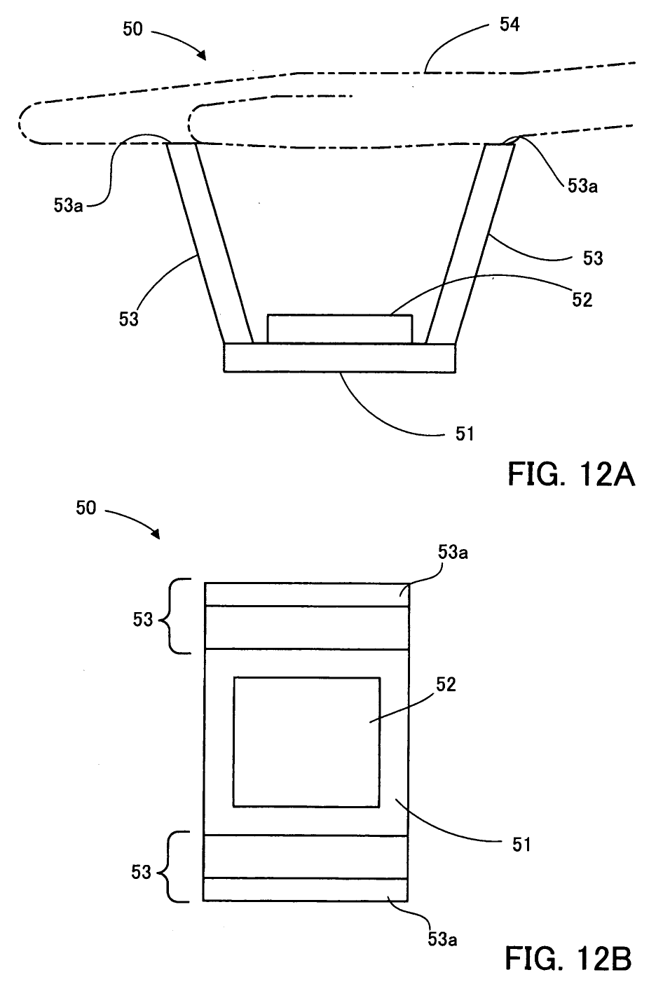

### DESCRIPTION OF THE PREFERRED EMBODIMENTS

The invention will now be described in detail with reference to drawings showing a preferred embodiments thereof.

### First Embodiment

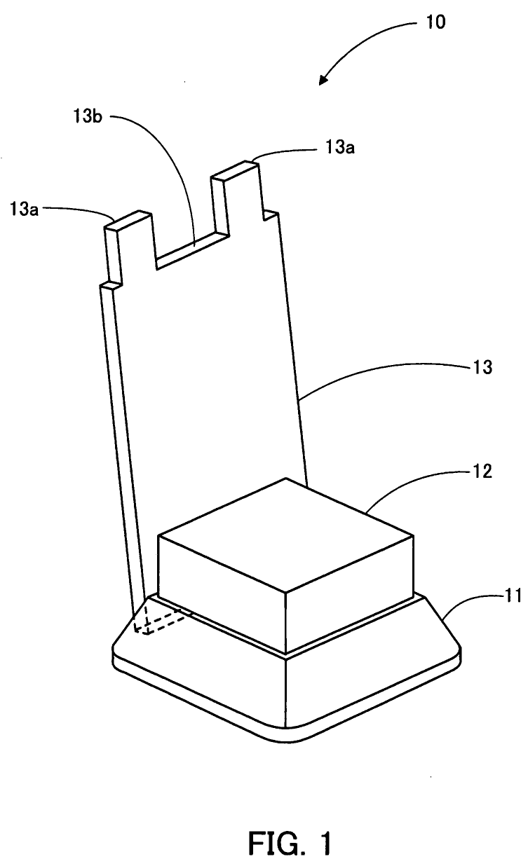

FIG. 1 is a perspective view of a vein imaging device according to the first embodiment of the present invention. The vein imaging device 10 includes a support member 11, an imaging section 12, and a guide member 13.

The support member 11 supports the imaging section 12 and the guide member 13.

The imaging section 12 is supported by the support member 11 by being removably inserted into the same. The imaging section 12 is formed e.g. by an imaging element for detecting incident near infrared rays, and images a vein pattern of a palm of a hand.

The guide member 13 is provided for guiding the position of a finger to be imaged and is formed to have a plate shape. When the vein imaging device 10 is in use, the guide member 13 is erected on a periphery of an imaging surface of the imaging section 12. In the present embodiment, the guide member 13 is supported by the support member 11 by being removably inserted into the same, by way of example. The guide member 13 has protrusions 13a and a finger-holding portion 13b arranged on an end opposite from the support member 11.

The protrusions 13a are formed by a pair of protrusions which protrude in the direction of height from the imaging surface of the imaging section 12, and are arranged to be spaced from each other in a direction parallel to the imaging surface, by a predetermined distance. The height of the protrusions 13a is set e.g. to 1 cm such that the position of the finger in the horizontal direction (i.e. in a parallel direction of the protrusions) is fixed.

The finger-holding portion 13b corresponds to a recess formed between the pair of protrusions 13a. The finger is inserted between the protrusions 13a from a surface of the guide member 13 on a side thereof toward the imaging section 12, and is brought into contact with the finger-holding portion 13b, whereby it is possible to guide the distance from the imaging section 12 to the palm and the position of the palm in the horizontal direction such that the distance and the position become optimum for imaging the vein pattern of the palm.

Further, the guide member 13 is formed such that it is inserted into the support member 11. An angle at which the guide member 13 is erected with respect to the support member is determined such that when the finger is inserted into the finger-holding portion 13b, the center of the palm is disposed exactly above the center of the imaging surface of the imaging section 12.

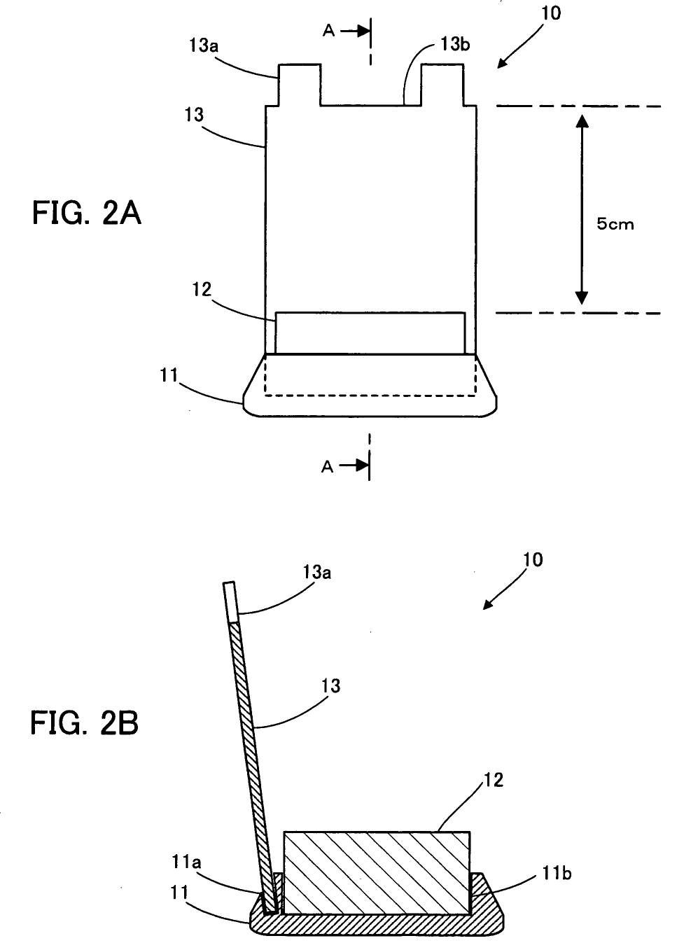

FIGS. 2A and 2B are views showing the construction of the vein imaging device according to the first embodiment, wherein FIG. 2A is a front view of the vein imaging device in use, and FIG. 2B is a cross-sectional view taken on line A-A of FIG. 2A.

As shown in FIG. 2A, the distance between the imaging section 12 and the finger-holding portion 13b is determined depending on the angle of view and focal distance of the imaging section 12. The distance between the imaging section 12 and the finger-holding portion 13b is 5 cm, for example. Further, FIG. 2B shows a state in which the imaging section 12 and the guide member 13 are removably inserted into the support member 11. The support member 11 includes a groove 11a and a grove 11b. The groove 11a is provided for inserting the guide member 13 therein. The groove 11b is provided for inserting the imaging section 12 therein. When storing the vein imaging device 10, it is possible to draw out the guide member 13 from the groove 11a to separate the support member 11 and the guide member 13 from each other, and to separate the imaging section 12 from the support member 11, as required, for storage.

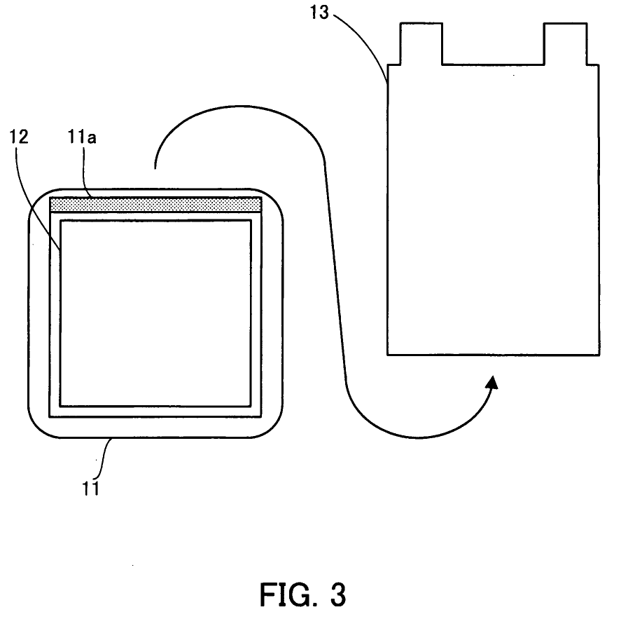

FIG. 3 is a view showing the support member and the guide member of the vein imaging device according to the first embodiment, in a state in which they are separated from each other. The guide member 13 inserted into the groove 11a of the support member 11 can be drawn out from the groove 11a, as shown in FIG. 3. The support member 11 and the guide member 13 can be stored in the state in which they are separated from each other. Further, in using the vein imaging device 10 for personal identification, the guide member 13 is inserted into the groove 11a.

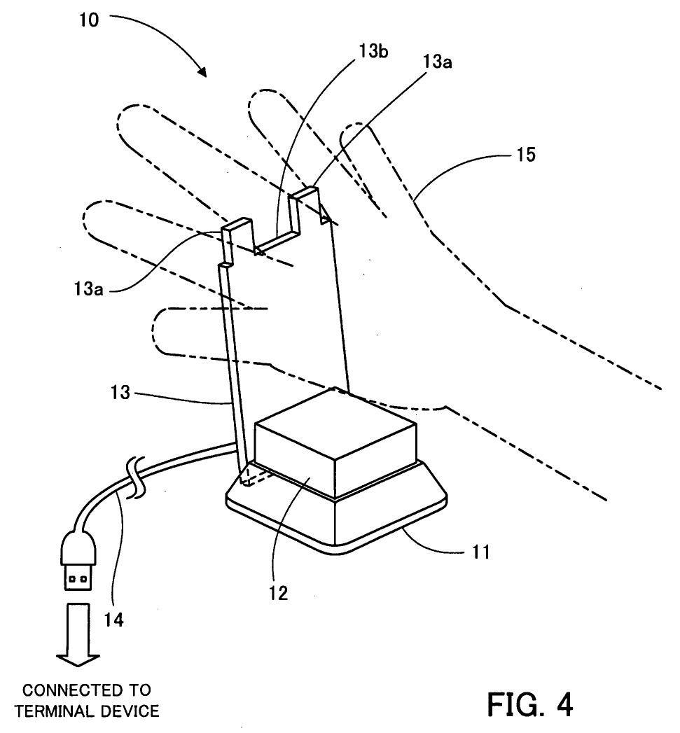

FIG. 4 is a schematic view showing an example of use of the vein imaging device according to the first embodiment. In FIG. 4, a hand 15 in a state in which fingers thereof are placed on the vein imaging device 10 is also shown. The vein imaging device 10 further includes an external connection connector 14.

The external connection connector 14 is connected to a terminal device (not shown), and a vein pattern of a palm of the hand 15, imaged by the vein imaging device 10, is transmitted to the terminal device. Then, personal identification by the vein pattern is performed using vein pattern comparison software installed in the terminal device. Alternatively, the personal identification is performed by analyzing and extracting feature information of the transmitted vein pattern using software (algorithm) installed in the terminal device, and comparing the extracted feature information and feature information registered in advance. The external connection connector 14 is implemented by a connector compliant with the USB (Universal Serial Bus) 2.0, for example. It should be noted that the vein imaging device 10 may have the comparison function. In this case, for example, only information indicative of the result of personal identification is transmitted to the terminal device by the external connection connector 14.

Further, by inserting a second finger of the hand 15 into the finger-holding portion 13b, the horizontal position of the palm of the hand 15 is guided such that it is suitable for imaging the vein pattern. Further, by setting the height of the finger-holding portion 13b to an optimum height for the imaging section 12 to image the vein pattern, the height of the palm of the hand 15 is guided to the height position of the finger-holding portion 13b.

As described above, no guide member for supporting a wrist of the hand 15 is provided, but the guide member 13 for placing fingers of the hand 15 thereon is inserted into the support member 11 which has approximately the same installation area as that of the imaging section 12, and hence it is possible to minimize the installation area of the vein imaging device 10 to achieve space saving. Further, since the guide member 13 is removably inserted into the support member 11, it is possible to separate the guide member 13 from the support member 11, as required. This makes it possible to lay the guide member 13 on its side for storage, and hence no space in the direction of the height of the guide member 13 is required, whereby it is possible to achieve space saving in storing the vein imaging device 10.

It should be noted that although in the above description, the guide member 13 is removably inserted into the support member 11, the guide member 13 may be pivotally inserted. In this case, when in use, the guide member 13 is set to a state erected with respect to the imaging surface of the imaging section 12, and when not in use, the guide member 13 is caused to fall toward the imaging surface such that it becomes parallel to the imaging surface, whereby it is possible to reduce a storage space required when the vein imaging device 10 is not used.

### Second Embodiment

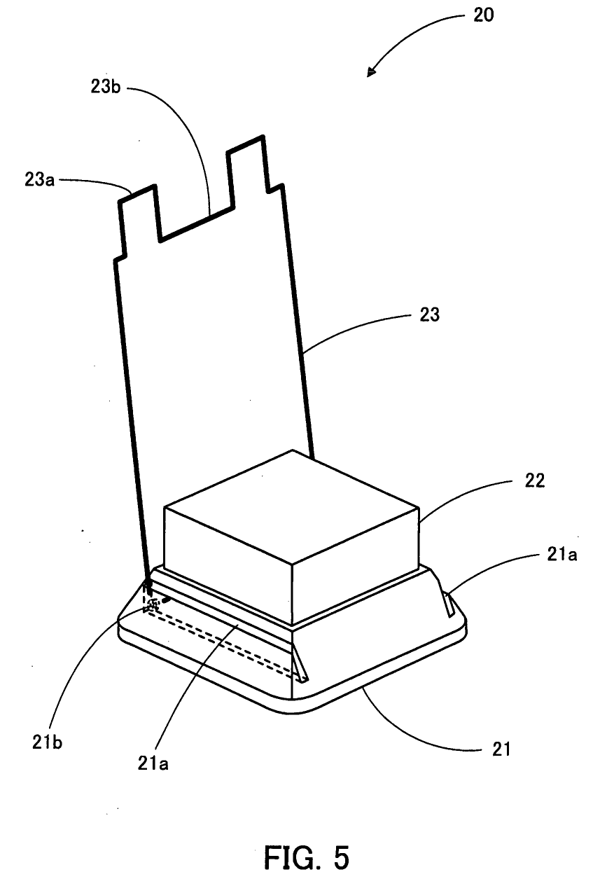

FIG. 5 is a perspective view of a vein imaging device according to a second embodiment of the present invention. The vein imaging device 20 according to the present embodiment is distinguished from the vein imaging device 10 according to the first embodiment in that the guide member 13 is replaced by one formed by a wire. The vein imaging device 20 includes a support member 21, an imaging section 22, and a guide member 23.

The support member 21 supports the imaging section 22 and the guide member 23. The support member 21 includes a pair of grooves 21a. The grooves 21a accommodate the guide member 23, and are formed in parallel with each other with the imaging section 22 interposed therebetween. Through holes 21b are provided for mounting the guide member 23 on the support member 21 by causing ends of the guide member 23 to extend therethrough.

The imaging section 22 corresponds to the imaging section 12 appearing in FIG. 1.

The guide member 23 is formed by bending a wire. In the illustrated example, the guide member 23 is formed such that an outer shape thereof exists on the same plane. When the vein imaging device 20 is used, the guide member 23 is erected on a periphery of the imaging surface of the imaging section 22. The guide member 23 is pivotally mounted on the support member 21, and is supported by one end walls of the grooves 21a at an optimum angle for the imaging section 22 to image the vein pattern. The other end of the guide member 23 is formed with protrusions 23a and a finger-holding portion 23b, and the one ends thereof form pivotal portions.

The protrusions 23a correspond to the protrusions 13a appearing in FIG. 1.

The finger-holding portion 23b correspond to the finger-holding portion 13b in FIG. 1.

The one ends of the guide member 23 extend horizontally through the pair of through holes 21b formed in the support member 21 such that the guide member 23 is pivotally movable about the through holes 21b.

Further, the angle of the guide member 23 with respect to the imaging surface of the imaging section 22 in the erected state of the guide member 23 is determined such that when fingers of a hand are placed on the guide member 23, the center of a palm of the hand is disposed exactly above the center of the imaging surface of the imaging section 22.

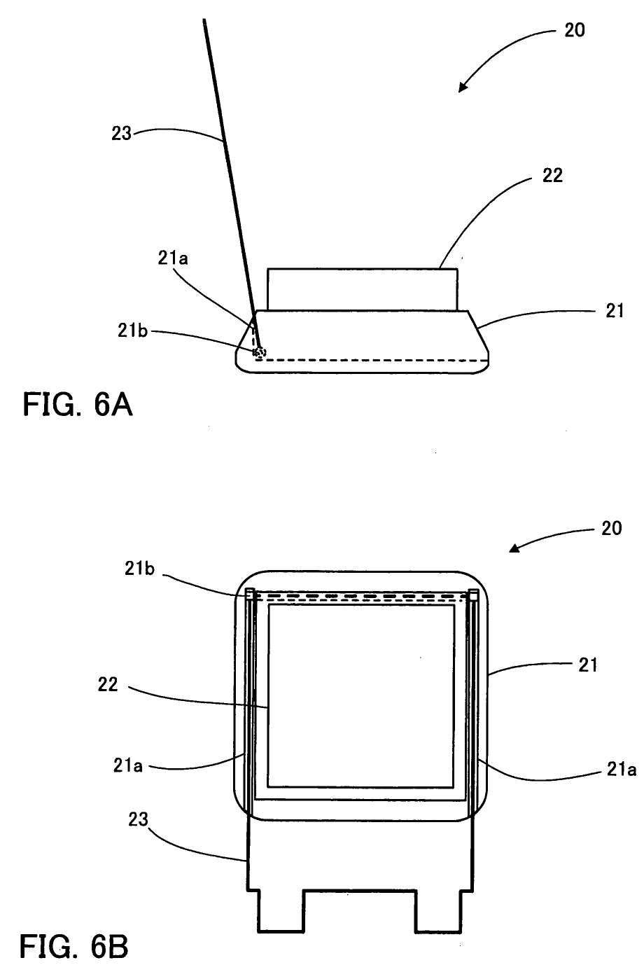

FIGS. 6A and 6B are views showing the construction of the vein imaging device according to the second embodiment, wherein FIG. 6A is a left side view of the vein imaging device in use, and FIG. 6B is a top view of the same in storage. As shown in FIG. 6A, the guide member 23 is supported by the end walls of the grooves 21a. Further, as shown in FIG. 6B, when the vein imaging device 20 is stored, it is possible to accommodate the guide member 23 in the grooves 21a by causing the guide member 23 to fall toward the imaging section 22 of the vein imaging device 20.

In the present embodiment, how to use the vein imaging device 20 is similar to that described as to the vein imaging device 10 in the first embodiment, and hence description of an example thereof is omitted.

As described above, no guide member for supporting a wrist of the hand is provided, but the guide member 23 for placing fingers thereon is inserted into the support member 21 which has approximately the same installation area as that of the imaging section 22, and hence it is possible to minimize the installation area of the vein imaging device 20 to achieve space saving. Further, since the guide member 23 is configured to be pivotally movable, it is possible to cause the guide member 23 to fall, as required. This makes it possible to lay the guide member 23 on its side for storage, and hence no space in the direction of the height of the guide member 23 is required for storage thereof, which makes it possible to achieve space saving in storing the vein imaging device 20.

It should be noted that although in the above description, the guide member 23 is assumed to be pivotally mounted on the support member 21, the guide member 23 may be removably inserted into the support member 21. This makes it possible to separate the support member 21 and the guide member 23 and store the vein imaging device 20 with the guide member 23 in a state laid on its side, and hence no space in the direction of the height of the guide member 23 is required for storage thereof, which makes it possible to achieve space saving in storing the vein imaging device 20.

### Third Embodiment

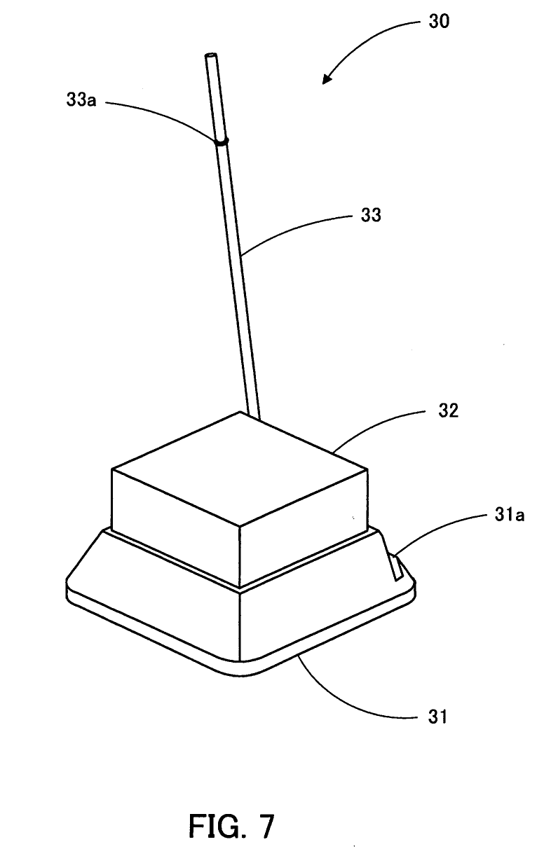

FIG. 7 is a perspective view of a vein imaging device according to a third embodiment of the present invention. The vein imaging device 30 includes a support member 31, an imaging section 32, and a guide member 33.

The support member 31 supports the imaging section 32 and the guide member 33. The support member 31 includes a groove 31a. The groove 31a accommodates the guide member 33.

The imaging section 32 corresponds to the imaging section 12 appearing in FIG. 1.

The guide member 33 is bar-shaped, and is erected on a periphery of an imaging surface of the imaging section 32 when the vein imaging device 30 is used. In the present embodiment, the guide member 33 is pivotally mounted on the support member 31, and is supported by an end wall of the groove 31a at an optimum angle for the imaging section 32 to image a vein pattern. The guide member 33 includes a height-indicating portion 33a.

The height-indicating portion 33a is a mark for indicating an optimum height position with respect to the imaging surface of the imaging section 32 when imaging a vein pattern. The height-indicating portion 33a guides a finger of the user to a position where the finger should be brought into contact with the guide member 33. By causing part of the finger to be brought into contact with the height-indicating portion 33a, the finger can be guided such that a height from the imaging section 32 to the palm is optimized for imaging the vein pattern. Further, by causing e.g. a base portion between a middle finger and a ring finger to be brought into contact with the height-indicating portion 33a from the side of the imaging section 32 of the vein imaging device 30, it is possible to guide the horizontal position of the hand. Further, since the guide member 33 is sandwiched between the middle finger and the ring finger, it is possible to prevent the hand from moving from the position to which the hand is guided.

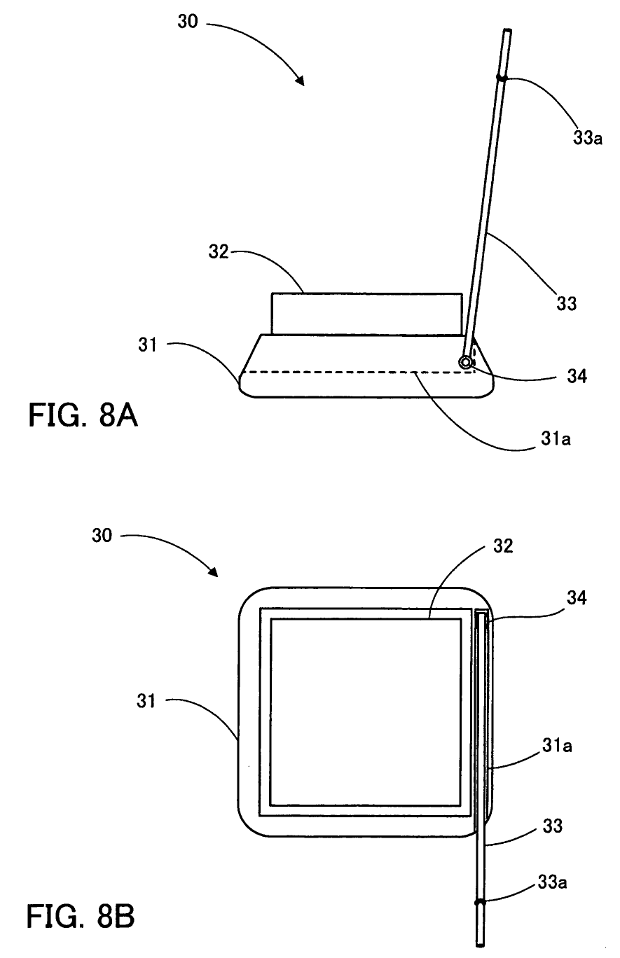

FIGS. 8A and 8B are views showing the construction of the vein imaging device according to the third embodiment, wherein FIG. 8A is a right side view of the vein imaging device in use, and FIG. 8B is a top view of the same in storage. The vein imaging device 30 further includes a pivotal portion 34.

For example, an end of the guide member 33 is caused to extend horizontally through the support member 31, whereby the pivotal portion 34 serves as a pivot about which the guide member 33 is pivotally moved.

As shown in FIG. 8A, the guide member 33 is supported by the end wall of the groove 31a. Further, as shown in FIG. 8B, when the vein imaging device 30 is stored, it is possible to accommodate the guide member 33 in the groove 31a by causing the guide member 33 to fall toward the imaging section 32 of the vein imaging device 30.

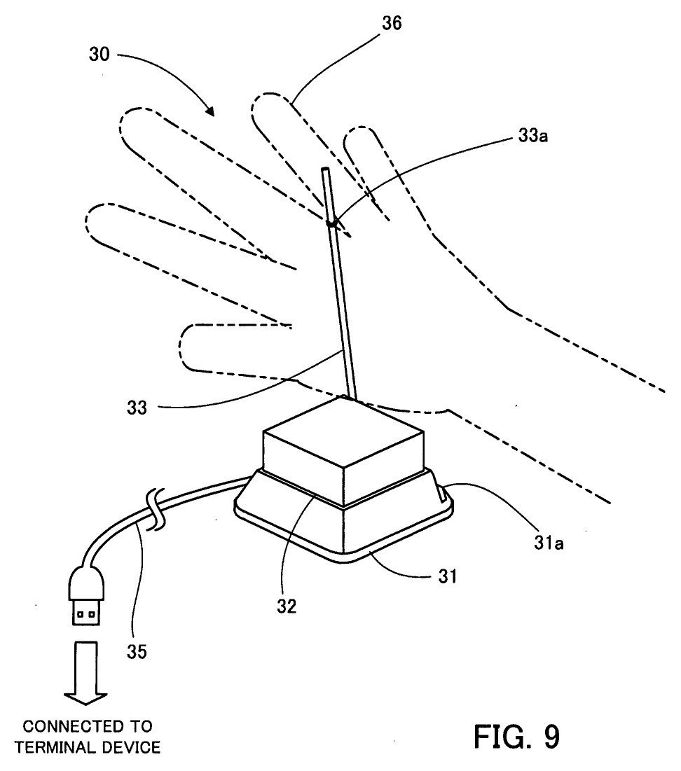

FIG. 9 is a schematic view showing an example of use of the vein imaging device according to the third embodiment. In FIG. 9, a hand 36 in a state in which fingers thereof are brought into contact with the vein imaging device 30 is also shown.

The vein imaging device 30 further includes an external connection connector 35. In the present embodiment, the external connection connector 35 corresponds to the external connection connector 14 appearing in FIG. 4.

The hand 36 has a base portion between a middle finger and a ring finger thereof brought into contact with the height-indicating portion 33a from the side of the imaging section 32 of the vein imaging device 30. The height of the height-indicating portion 33a is set to an optimum height for the imaging section 32 to image the vein pattern, whereby the height of the hand 36 can be guided to this position of the optimum height. Further, simultaneously, the horizontal position of the hand 36 can be guided to an optimum position for the imaging section 32 to image the vein pattern. Further, since the guide member 33 is sandwiched between the middle finger and the ring finger, it is possible to prevent the hand 36 from moving from the position to which the hand 36 is guided.

As described above, no guide member for supporting a wrist of the hand 36 is provided, but the guide member 33 which is brought into contact with fingers is mounted on the support member 31 which has approximately the same installation area as that of the imaging section 32, and hence it is possible to minimize the installation area of the vein imaging device 30 to achieve space saving. Further, since the guide member 33 is configured to be pivotally movable, it is possible to cause the guide member 33 to fall, as required. This makes it possible to store the vein imaging device 30 with the guide member 33 fallen toward the imaging section 32, and hence no space in the direction of the height of the guide member 33 is required for storage thereof, which makes it possible to achieve space saving in storing the vein imaging device 30.

It should be noted that although in the above description, the guide member 33 is assumed to be pivotally mounted on the support member 31, the guide member 33 may be removably inserted into the support member 31. This makes it possible to separate the support member 31 and the guide member 33 and store the guide member 33 in a state laid on its side, and hence no space in the direction of the height of the guide member 33 is required for storage thereof, which makes it possible to achieve space saving in storage.

### Fourth Embodiment

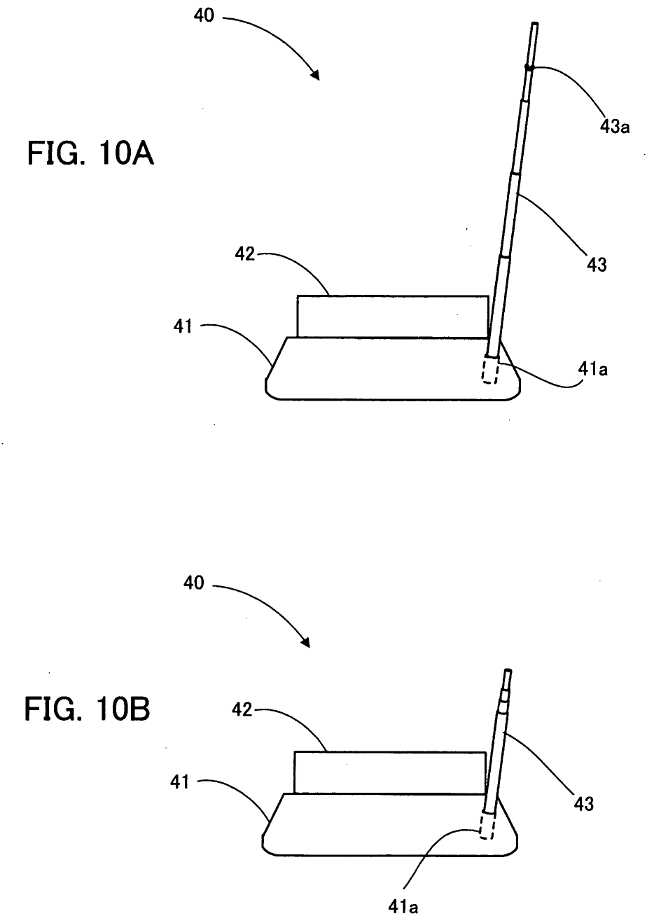

FIGS. 10A and 10B are views showing the construction of a vein imaging device according to a fourth embodiment, wherein FIG. 10A is a right side view of the vein imaging device in use, and FIG. 10B is a right side view of the same in storage. The vein imaging device 40 according to the present embodiment is distinguished from the third embodiment in that the bar-shaped guide member 33 is replaced by one configured to be telescopic (capable of extending and contracting). The vein imaging device 40 includes a support member 41, an imaging section 42, and a guide member 43.

The support member 41 supports the imaging section 42 and the guide member 43. The support member 41 includes a groove 41a. The groove 41a is provided for inserting the guide member 43 therein.

The imaging section 42 corresponds to the imaging section 12 appearing in FIG. 1.

The guide member 43 is in the form of a telescopic bar. The guide member 43 is erected on a periphery of an imaging surface of the imaging section 42. The guide member 43 is removably inserted into the support member 41, and is supported by an end wall of the groove 41a at an optimum angle for the imaging section 42 to image a vein pattern.

A height-indicating portion 43a corresponds to the height-indicating portion 33a appearing in FIG. 7.

In the present embodiment, as shown in FIG. 10B, when the vein imaging device 40 is stored, the length of the guide member 43 can be reduced. This makes space in the direction of the height of the guide member 43 unnecessary for storage thereof, whereby it is possible to achieve space saving. It should be noted that since the guide member 43 is removably inserted, and hence if the guide member 43 is stored in a contracted state after separating the same from the support member 41, it is possible to move effectively achieve space saving.

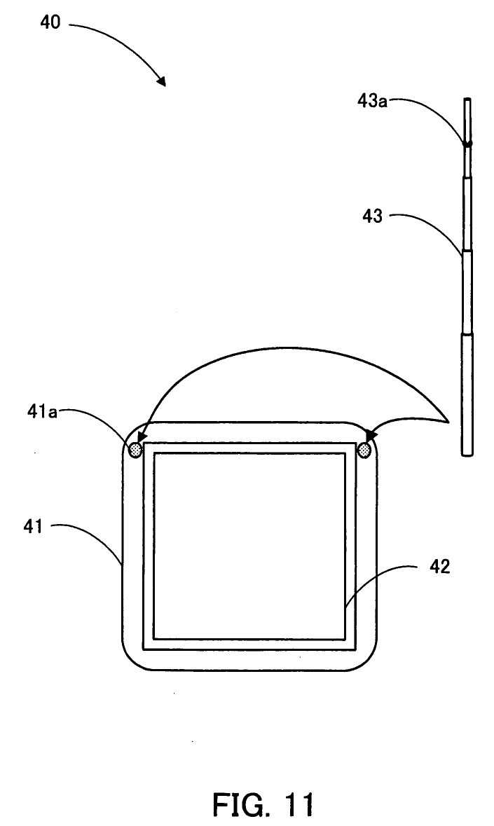

FIG. 11 is a top view of the vein imaging device according to the fourth embodiment. As shown in FIG. 11, by providing the groove 41a on each of the left and right sides of the support member 41, as viewed from the side of the imaging section 42 of the vein imaging device 40, the guide member 43 can be erected at a location desired by the user. For example, when vein authentication is desired to be performed by using a left hand, or by bringing a base portion between a forefinger and a middle finger of a right hand into contact with the guide member 43, the guide member 43 can be erected on the left side of the support member 41, as viewed from the side of the imaging section 42 of the vein imaging device 40.

In the present embodiment, how to use the vein imaging device 40 is similar to that described as to the vein imaging device 30 in the third embodiment, and hence description of an example thereof is omitted.

As described above, no guide member for supporting a wrist of a hand is provided, but the guide member 43 which is brought into contact with fingers is inserted into the support member 41 which has approximately the same installation area as that of the imaging section 42, and hence it is possible to minimize the installation area of the vein imaging device 40 to achieve space saving. Further, since the guide member 43 is configured to be removably inserted, it is possible to separate the guide member 43 from the support member 41, as required. This makes it possible to save the vein imaging device 40 with the guide member 43 laid on its side, and hence no space in the direction of the height of the guide member 43 is required for storage thereof, whereby it is possible to save the storage space for the vein imaging device 40. In the present embodiment, since the guide member 43 is telescopic, the guide member 43 can be stored in a contracted state after separating the same from the support member 41.

It should be noted that although in the above description, the guide member 43 is assumed to be removably inserted, the guide member 43 may be pivotally mounted on the support member 41. Even when the guide member 43 is pivotally mounted on the support member 41, it is possible to store the vein imaging device 40 after reducing the length of the guide member 43 and causing the same to be laid on its side. Therefore, no space in the direction of the guide member 43 is required for storage thereof, which make it possible to achieve space saving for storing the vein imaging device 40.

According to the above-described vein imaging device, it is possible to achieve space saving.

The foregoing is considered as illustrative only of the principles of the present invention. Further, since numerous modifications and changes will readily occur to those skilled in the art, it is not desired to limit the invention to the exact construction and applications shown and described, and accordingly, all suitable modifications and equivalents may be regarded as falling within the scope of the invention in the appended claims and their equivalents.

What is claimed is:

1. A vein imaging device that images a vein pattern of a palm, comprising:
an imaging section that images the vein pattern; and
a guide member that is erected on a periphery of an imaging surface of said imaging section,
wherein said guide member guides a position of the palm with respect to the imaging surface by having a finger brought into contact therewith at a predetermined height position from said imaging surface of said imaging section.

2. The vein imaging device according to claim 1, wherein said guide member includes a pair of protrusions that protrude in a direction of height from said imaging surface, and are arranged to be spaced from each other in a direction parallel to said imaging surface, by a predetermined distance, said guide member guiding horizontal and vertical positions of the palm with respect to said imaging surface, by having the finger inserted from a side of said imaging section into a recess formed between said pair of protrusions so as to be brought into contact therewith.

3. The vein imaging device according to claim 2, wherein said guide member is formed to be plate-shaped or wire-shaped.

4. The vein imaging device according to claim 3, further comprising a support member that supports said imaging section, and
wherein said guide member is removably inserted into said support member or pivotally mounted on said support member.

5. The vein imaging device according to claim 3, further comprising a support member that supports said imaging section, and
wherein said guide member is pivotally mounted on said support member, and is disposed to extend along said imaging surface when the vein imaging device is stored.

6. The vein imaging device according to claim 1, further comprising a support member that supports said imaging section, and
wherein said guide member is bar-shaped, and has a mark that indicates the position for use in having the finger brought into contact therewith, said guide member being removably inserted into said support member or pivotally mounted on said support member.

7. The vein imaging device according to claim 6, wherein said guide member is capable of extending and contracting.
